(19) United States
(12) Patent Application Publication
(10) Pub. No.: US 2022/0326252 A1
(43) Pub. Date: Oct. 13, 2022

(54) ELECTRON TRANSFER DISSOCIATION AND MASS SPECTROMETRY FOR IMPROVED PROTEIN SEQUENCING OF MONOCLONAL ANTIBODIES

(71) Applicant: Regeneron Pharmaceuticals, Inc., Tarrytown, NY (US)

(72) Inventors: Xiaojing Zheng, Croton-on-Hudson, NY (US); Milos Cejkov, Ridgewood, NJ (US); Tyler Greer, Elmsford, NY (US); Reid O'Brien Johnson, Hartsdale, NY (US)

(21) Appl. No.: 17/681,156

(22) Filed: Feb. 25, 2022

Related U.S. Application Data
(60) Provisional application No. 63/153,886, filed on Feb. 25, 2021.

(51) Int. Cl. G01N 33/68; G01N 30/72

(52) U.S. Cl. CPC G01N 33/6848; G01N 30/7233; G01N 33/6854

(57) ABSTRACT

The present disclosure provides an improved method for accurately measuring the amino acid sequence of a therapeutic protein, in particular, an antibody, to insure the homogeneity of the protein such that it is suitable for administering to a human subject for treating a disease or disorder. The methods employ analytical physical chemistry techniques, in particular, a guided electron-transfer dissociation (ETD) and mass spectrometry (MS) approach for robust sequence coverage of the molecule with high accuracy and speed of use.

### CROSS-REFERENCE TO RELATED APPLICATION

This application claims priority to and the benefit of U.S. Provisional Patent Application No. 63/153,886, filed Feb. 25, 2021 which is herein incorporated by reference.

### SEQUENCE LISTING

The instant application contains a Sequence Listing which has been submitted electronically in ASCII format and is hereby incorporated by reference in its entirety. Said ASCII copy, created on Jun. 30, 2022, is named 070816-02161_SL.txt and is 12,657 bytes in size.

### FIELD

Aspects of the present invention are generally directed to methods for characterizing antibody sequence fidelity. More specifically, the present disclosure provides analytical physical chemistry techniques, protein fragment selection criteria, and multivariate analysis to achieve higher sequence accuracy and coverage using electron transfer dissociation (ETD) and mass spectrometry (MS).

### BACKGROUND

Monoclonal antibody (mAb) therapeutics, due to their high specificity and preferable pharmacokinetic properties, are poised to remain a major force in the biopharmaceutical market (Walsh, 2018, Nat Biotechnol, 36:1136-1145; Kaplon et al., 2020, Mabs, 12:1703531; Mould and Meibohm, 2016, BioDrugs, 30:275-293). Due to the importance of glycosylation and disulfide bonds on the proper function of monoclonal antibodies (mAbs), mAbs are often produced in mammalian cell systems.

However, in addition to applying modifications needed for mAb function, production in mammalian cell systems is known to introduce product heterogeneity (Meibohm et al., 2019, Pharmaceutical Biotechnology Fundamentals and Applications, 5^{th} ed.). Because post-translational modification (PTM) micro heterogeneity can impact important quality attributes of mAb therapeutics, including stability, solubility, and pharmacodynamic and pharmacokinetic properties, regulatory agencies require extensive analytical characterization to ensure product quality and consistency (Meibohm et al.).

Mass spectrometry (MS) techniques, specifically top-down and bottom-up MS, have proven to be powerful tools in the structural characterization of antibodies. Each approach provides unique information regarding protein structure and potential post-translational modifications (Fornelli et al., 2018, Anal Chem, 90:8421-8429; Wang et al., 2019, Sci Rep, 9:2345). Mass spectrometry may be used in combination with liquid chromatography, for example liquid chromatography-mass spectrometry (LC-MS) or liquid chromatography-tandem mass spectrometry (LC-MS² or LC-MS/MS), to further enhance the ability to characterize analytes, for example antibodies.

Top-down MS combines intact mass analysis with tandem mass spectrometry (M5²), does not require enzymatic digestion, and provides a wealth of information regarding, for example, post-translational modifications, protein structure, and drug-target interactions (Donnelly et al., 2019, Nat Methods, 16:587-594). However, top-down MS² of large biopharmaceuticals like antibodies has limitations. Chief among these is the prodigious size of large molecules that limits the sequence coverage possible without any enzyme treatment (Fornelli et al.).

Bottom-up MS is an alternative approach that can provide sequence coverage with amino acid-level resolution. One drawback of this technique is that the high sequence coverage comes at the cost of extensive sample preparation, including, for example, protein denaturation, disulfide reduction, cysteine alkylation, and/or enzymatic digestion of the protein of interest (Nielsen et al., 2008, Nat Methods, 5:459-460; Lippincott et al., 1999, Anal Biochem, 267:57-64). Bottom-up MS is not only labor-intensive, but it also can introduce chemical modification artifacts during sample preparation. Because of this risk, bottom-up MS requires extensive development to produce an optimized method that minimizes sample preparation-induced artifacts.

Middle-down MS is an alternative approach to antibody characterization that combines the benefits of top-down and bottom-up MS analysis (Chait, 2006, Science, 314:65-66; Fornelli et al., 2014, Anal Chem, 86:3005-3012; Tsybin et al., 2011, Anal Chem, 83:8919-8927; Fornelli et al., 2012, Mol Cell Proteomics, 11:1758-1767). Middle-down MS analysis often uses the immunoglobulin G-degrading enzyme of S. pyogenes (IdeS) to cleave a monoclonal antibody (mAb) into its subunits. Middle-down MS analysis of mAb subunits can be coupled with electron-transfer dissociation (ETD), because large subunits generated with limited protease digestion possess greater than a +3 charge, which is preferential for ETD fragmentation analysis. These highly charged gas phase precursor ions are fragmented with ETD to produce a c- and z-ion series, which reveals structural information about the protein of interest, such as amino acid sequence and PTM identities (Mikesh et al., 2006, Biochim Biophys Acta, Proteins Proteomics, 1764:1811-1822; Syka et al., 2004, PNAS, 101:9528-9533; Zhurov et al., 2013, Chem Soc Rev, 42:5014-5030).

Because fragmentation of precursor ions by ETD utilizes low energy electrons during ion-ion interactions and proceeds via an exothermic process to induce backbone cleavage, labile PTMs are preserved in the MS² spectra, allowing for their identification and localization to a specific amino acid (Brodbelt, 2016, Anal Chem, 88:30-51). Electron-transfer dissociation fragmentation of intact monoclonal antibodies was first reported by Tsybin et al., while ETD fragmentation of subunits of monoclonal antibodies was first presented by Fornelli et al.

Previous work on middle-down analysis using ETD demonstrated that sequence coverage of up to 49% could be achieved for the Fc/2 subunit per reversed-phase liquid chromatography mass spectrometry (RPLC-MS) run, and increased this coverage up to 68% by combining at least six ETD runs (Fornelli et al. 2014). More recently, coverage was shown to be further improved to 87% by integrating three ETD runs (59.8%), one electron transfer higher-energy collisional dissociation (EThcD) run (51.7%) and two ultraviolet photodissociation (UVPD) runs (61.2%) into the analysis, combining multiple fragmentation techniques (Fornelli et al. 2018; Fornelli et al. 2014).

Since mass spectrometers currently on the market are built for a wide range of applications, extensive instrument parameter optimization is required to find optimal parameters for a specific application. An unmet need is a method to determine optimal parameters for maximizing ETD MS² sequence coverage of mAb subunits.

Advancements in mass spectrometers and fragmentation methods, including hybrid fragmentation methods and UVPD, have proportionally increased the number of instrument parameters an analyst should consider. With many different approaches and parameters to increase sequence coverage of antibody subunits, finding an optimal set of conditions can be a major challenge. For ETD, the interactions between many available parameters and how they impact charge reduction, fragmentation efficiency, and quality of MS² spectra are often overlooked, and settings typically used for analysis are often based on established theory, previous experience, or published methods. More recent mass spectrometers, like the Orbitrap Fusion Lumos Tribrid, contain sophisticated calibration routines for ETD, but they are performed using relatively small analytes like angiotensin and are not representative of the larger ions fragmented during top- or middle-down analysis of large proteins. Furthermore, these routines do not account for all sources of instrument drift that could affect ETD fragment production and their ensuing impact on optimal parameters.

Hence, there is a need for a systematic approach to optimize instrument ETD operating conditions that would provide maximum MS² sequence coverage, and that can be applied to all mass spectrometers.

Accordingly, it would be desirable to provide a simple method for accurate and precise sequencing of a protein, in particular, antibodies and antibody subunits.

### SUMMARY

Therefore, an object of the present invention is to employ a statistical design of experiment (DOE) approach that can be used to determine optimal electron-transfer dissociation (ETD) parameters to maximize sequence coverage of monoclonal antibody subunits. In addition, this method can be applied to an array of therapeutic monoclonal antibodies, to determine molecule-specific parameters, because each antibody may fragment slightly differently during electron-transfer dissociation (ETD) experiments.

In particular, the present invention provides methods useful for accurate de novo sequencing of proteins, such as antibodies or antibody subunits using ETD and MS, for example LC-MS². The methods are informed by DOE such that D-optimal instrument settings are identified and selected.

One approach to find optimal ETD MS² parameters is to utilize a design of experiments (DOE), which allows for screening of different combinations of parameters to find the ones that provide maximum sequence coverage of a subunit. Rather than evaluating “one-factor at a time” (OFAT), which is inefficient considering the number of MS instrument parameters, DOE allows for simultaneous evaluation of the instrument's many different parameters.

Specifically, the m/z (mass-to-charge ratio) isolation window, ETD reaction time, ETD reagent target, and MS² AGC target (tandem mass spectrometry with automatic gain control) are important parameters affecting ETD MS² spectral quality and therefore can impact the overall MS² subunit sequence coverage.

To decrease the workload and still have a meaningful model, a D-optimal design was adopted. D-optimal design is a computer-assisted design, wherein a subset of all possible combinations is chosen with a goal of maximizing D-efficiency of the DOE (de Aguiar et al., 1995, Chemom Intell Lab Syst, 30:199-210). The D-optimal design can be applied to ETD to extrapolate relevant parameters that control ETD MS² sequence coverage (using as a basis the group of parameters selected by the user that affect the selected outcome), estimate the effect size of each parameter and interaction, predict the response resulting from these chosen parameter combinations, and ultimately determine improved operating conditions to achieve maximum coverage (Randall et al., 2013, J Am Soc Mass Spectrom, 24:1501-1512; Kelstrup et al., 2012, J Proteom Res, 11:3487-3497; Sun et al., 2013, Rapid Commun Mass Spectrom, 27:157-162; Coffey and Yang, 2018, Statistics for Biotechnology Process Development).

Therefore, DOE is a powerful mathematical tool that can be applied using the method of the present invention in order to improve sequence coverage of a polypeptide, for example a mAb or a mAb subunit, in particular when applied to ETD MS² analysis.

The present invention also provides proteins, for example antibodies, variants, or antibody fusions, that have been sequenced according to the methods of the present invention.

Advantages of the present invention include, but are not limited to, a robust and highly accurate assay using analytical physical chemistry for high speed and accurate de novo sequencing of proteins, for example, therapeutic antibodies; therapeutic antibodies produced to a higher level of sequence confidence as a result of the above as part of the manufacturing train; wide application for perfecting the manufacture of antibodies in clinical development and in commercial use; higher sequence coverage of monoclonal antibody subunits than any published ETD approach; higher sequence coverage with PTM information intact without sample artifacts, i.e., labile PTMs are preserved; higher sequence coverage (SC), for example, 19-26% improvement over known methods and the potential to reach 100% SC; and minimal steps, ease of use, and lower cost.

This disclosure provides a method for improving sequence coverage of a polypeptide. In some exemplary embodiments, the method comprises (a) selecting at least two parameters for tandem mass spectrometry that affect sequence coverage; and (b) using D-optimal design of experiments to determine a value of each of said at least two parameters, wherein said value is selected based on maximizing sequence coverage.

In one aspect, said polypeptide is an antibody, a bispecific antibody, a monoclonal antibody, a fusion protein, an antibody-drug conjugate, an antibody fragment, an antibody subunit, a host cell protein, a protein pharmaceutical product, or a digested fragment thereof.

In one aspect, the method further comprises carrying out the method in sequence or in parallel for two or more subunits of a polypeptide. In specific aspect, said subunits are selected from a group including an Fc/2, Fd, or LC subunit of an antibody.

In one aspect, said tandem mass spectrometry is middle-down mass spectrometry.

In one aspect, said mass spectrometer is an electrospray ionization mass spectrometer, nano-electrospray ionization mass spectrometer, or an Orbitrap-based mass spectrometer.

In one aspect, said tandem mass spectrometry includes electron-transfer dissociation, collision-induced dissociation, electron-transfer/collision-induced dissociation, electron-transfer/higher-energy collisional dissociation, ultra-violet photodissociation, or a combination thereof. In another aspect, said tandem mass spectrometry includes automatic gain control.

In one aspect, said mass spectrometer is coupled to a liquid chromatography system. In a specific aspect, said liquid chromatography system comprises reversed-phase liquid chromatography, ion exchange chromatography, size exclusion chromatography, affinity chromatography, hydrophobic interaction chromatography, hydrophilic interaction chromatography, mixed-mode chromatography, or a combination thereof.

In one aspect, said at least two parameters are selected from a group including m/z isolation window, ETD reaction time, ETD reagent target, MS² AGC target, and any combination thereof.

This disclosure also provides a method for determining an amino acid sequence of a polypeptide. In some exemplary embodiments, the method comprises (a) determining a value of at least two parameters for tandem mass spectrometry for a polypeptide using D-optimal design of experiments, wherein said value is selected based on maximizing sequence coverage; and (b) subjecting said polypeptide to tandem mass spectrometry analysis using said values of said at least two parameters to determine an amino acid sequence of said polypeptide.

In one aspect, said polypeptide is an antibody, a bispecific antibody, a monoclonal antibody, a fusion protein, an antibody-drug conjugate, an antibody fragment, an antibody subunit, a host cell protein, a protein pharmaceutical product, or a digested fragment thereof.

In one aspect, the method further comprises carrying out the method in sequence or in parallel for two or more subunits of a polypeptide. In a specific aspect, said subunits are selected from a group including an Fc/2, Fd, or LC subunit of an antibody.

In one aspect, said tandem mass spectrometry is middle-down mass spectrometry.

In one aspect, said mass spectrometer is an electrospray ionization mass spectrometer, nano-electrospray ionization mass spectrometer, or an Orbitrap-based mass spectrometer. In another aspect, said tandem mass spectrometry includes electron-transfer dissociation, collision-induced dissociation, electron-transfer/collision-induced dissociation, electron-transfer/higher-energy collisional dissociation, ultra-violet photodissociation, or a combination thereof. In a further aspect, said tandem mass spectrometry includes automatic gain control.

In one aspect, said mass spectrometer is coupled to a liquid chromatography system. In a specific aspect, said liquid chromatography system comprises reversed-phase liquid chromatography, ion exchange chromatography, size exclusion chromatography, affinity chromatography, hydrophobic interaction chromatography, hydrophilic interaction chromatography, mixed-mode chromatography, or a combination thereof.

In one aspect, said at least two parameters are selected from a group including m/z isolation window, ETD reaction time, ETD reagent target, MS² AGC target, and any combination thereof.

In one aspect, the method further comprises subjecting said polypeptide to enzymatic digestion prior to tandem mass spectrometry analysis. In a specific aspect, said enzymatic digestion comprises contacting said polypeptide to IdeS. In another aspect, the method further comprises subjecting said polypeptide to reduction prior to tandem mass spectrometry analysis.

In one aspect, the method further comprises carrying out the method independently two or more times and combining identified fragments from each tandem mass spectrometry analysis to determine an amino acid sequence of said polypeptide.

This disclosure provides an additional method for improving sequence coverage of a polypeptide. In some exemplary embodiments, the method comprises (a) selecting at least two parameters for tandem mass spectrometry that affect the number of fragments of each of a selection of different fragment sizes; and (b) using D-optimal design of experiments to determine a value of each of said at least two parameters, wherein said value is selected based on producing the greatest number of fragments for each of said selected fragment sizes.

In one aspect, said polypeptide is an antibody, a bispecific antibody, a monoclonal antibody, a fusion protein, an antibody-drug conjugate, an antibody fragment, an antibody subunit, a host cell protein, a protein pharmaceutical product, or a digested fragment thereof.

In one aspect, the method further comprises carrying out the method in sequence or in parallel for two or more subunits of a polypeptide. In specific aspect, said subunits are selected from a group including an Fc/2, Fd, or LC subunit of an antibody.

In one aspect, said tandem mass spectrometry is middle-down mass spectrometry.

In one aspect, said mass spectrometer is an electrospray ionization mass spectrometer, nano-electrospray ionization mass spectrometer, or an Orbitrap-based mass spectrometer.

In one aspect, said tandem mass spectrometry includes electron-transfer dissociation, collision-induced dissociation, electron-transfer/collision-induced dissociation, electron-transfer/higher-energy collisional dissociation, ultra-violet photodissociation, or a combination thereof. In another aspect, said tandem mass spectrometry includes automatic gain control.

In one aspect, said mass spectrometer is coupled to a liquid chromatography system. In a specific aspect, said liquid chromatography system comprises reversed-phase liquid chromatography, ion exchange chromatography, size exclusion chromatography, affinity chromatography, hydrophobic interaction chromatography, hydrophilic interaction chromatography, mixed-mode chromatography, or a combination thereof.

In one aspect, said at least two parameters are selected from a group including m/z isolation window, ETD reaction time, ETD reagent target, MS² AGC target, and any combination thereof.

In one aspect, said fragment sizes are selected from a group including fragments below about 5,000 Da, fragments between about 5,000 Da and about 10,000 Da, and fragments larger than about 10,000 Da.

This disclosure further provides another method for determining an amino acid sequence of a polypeptide. In some exemplary embodiments, the method comprises (a) determining a value of at least two parameters for tandem mass spectrometry for a polypeptide using D-optimal design of experiments, wherein said value is selected based on producing the greatest number of fragments for each of a selection of different fragment sizes; (b) subjecting said polypeptide to tandem mass spectrometry analysis using said values of said at least two parameters for each selected fragment size; and (c) combining identified fragments from said tandem mass spectrometry analysis using said values of said at least two parameters for each selected fragment size to determine an amino acid sequence of said polypeptide.

In one aspect, said polypeptide is an antibody, a bispecific antibody, a monoclonal antibody, a fusion protein, an antibody-drug conjugate, an antibody fragment, an antibody subunit, a host cell protein, a protein pharmaceutical product, or a digested fragment thereof.

In one aspect, the method further comprises carrying out the method in sequence or in parallel for two or more subunits of a polypeptide. In a specific aspect, said subunits are selected from a group including an Fc/2, Fd, or LC subunit of an antibody.

In one aspect, said tandem mass spectrometry is middle-down mass spectrometry.

In one aspect, said mass spectrometer is an electrospray ionization mass spectrometer, nano-electrospray ionization mass spectrometer, or an Orbitrap-based mass spectrometer. In another aspect, said tandem mass spectrometry includes electron-transfer dissociation, collision-induced dissociation, electron-transfer/collision-induced dissociation, electron-transfer/higher-energy collisional dissociation, ultra-violet photodissociation, or a combination thereof. In a further aspect, said tandem mass spectrometry includes automatic gain control.

In one aspect, said mass spectrometer is coupled to a liquid chromatography system. In a specific aspect, said liquid chromatography system comprises reversed-phase liquid chromatography, ion exchange chromatography, size exclusion chromatography, affinity chromatography, hydrophobic interaction chromatography, hydrophilic interaction chromatography, mixed-mode chromatography, or a combination thereof.

In one aspect, said at least two parameters are selected from a group including m/z isolation window, ETD reaction time, ETD reagent target, MS² AGC target, and any combination thereof.

In one aspect, the method further comprises subjecting said polypeptide to enzymatic digestion prior to tandem mass spectrometry analysis. In a specific aspect, said enzymatic digestion comprises contacting said polypeptide to IdeS. In another aspect, the method further comprises subjecting said polypeptide to reduction prior to tandem mass spectrometry analysis.

In one aspect, the method further comprises carrying out the method independently two or more times and combining identified fragments from each tandem mass spectrometry analysis to determine an amino acid sequence of said polypeptide.

This disclosure provides a further method for determining an amino acid sequence of an antibody. In some exemplary embodiments, the method comprises (a) selecting at least two parameters for ETD-MS² that affect sequence coverage for each subunit of an antibody, wherein said subunits include Fd, Fc/2 and LC; (b) using D-optimal design of experiments to determine a value of each of said at least two parameters for each of said subunits, wherein said value is selected based on maximizing sequence coverage of said subunit; (c) contacting said antibody to IdeS and a reducing agent to produce said subunits; (d) subjecting each of said subunits to ETD-MS² analysis using said values of said at least two parameters to identify amino acid sequences of fragments of each of said subunits; (e) independently repeating step (d) at least one more time to identify amino acid sequences of additional fragments of each of said subunits; and (f) combining said amino acid sequences of said fragments of (d) and (e) to determine an amino acid sequence of said antibody.

This disclosure provides another method for determining an amino acid sequence of an antibody. In some exemplary embodiments, the method comprises (a) selecting at least two parameters for ETD-MS² that affect the number of small, medium, and large fragments of subunit of an antibody, wherein said subunits include Fd, Fc/2 and LC, said small fragments consist of fragments smaller than about 5,000 Da, said medium fragments consist of fragments between about 5,000 Da and about 10,000 Da, and said large fragments consist of fragments greater than 10,000 Da; (b) using D-optimal design of experiments to determine a value of each of said at least two parameters for each of said fragment sizes for each of said subunits, wherein said value is selected on the basis of producing the greatest number of fragments of said size for said subunit; (c) contacting said antibody to IdeS and a reducing agent to produce said subunits; (d) subjecting each of said subunits to ETD-MS² analysis using said values of said at least two parameters for each of said fragment sizes to identify amino acid sequences of fragments of each of said subunits; (e) independently repeating step (d) at least one more time to identify amino acid sequences of additional fragments of each of said subunits; and (f) combining said amino acid sequences of said fragments of (d) and (e) to determine an amino acid sequence of said antibody.

These, and other, aspects of the present invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 shows a schematic of the assay of the present invention having four (4) steps as indicated. Step 1 shows a digestion step mediated by the IdeS enzyme such that three (3) 25 kilodalton (kD) polypeptides result, and they are Fd (heavy chain variable region of the antibody), Fc/2 (Fc fragment of the constant region of the antibody), and LC (the light chain variable region of the antibody), as indicated. Step 2 shows an exemplary DOE modeling for optimal use of ETD for sequence coverage of the above-mentioned antibody fragments. Step 3 shows an exemplary application of three different settings that can be applied within the DOE envelope. Step 4 shows an exemplary output of improved sequence coverage (SC) of an antibody subunit wherein a majority of residues are accurately identified. FIG. 1 discloses SEQ ID NO: 3.

FIG. 2A shows a comparison of predicted and observed sequence coverage of Fc/2, LC, and Fd subunits of an antibody using the method of the present invention, according to an exemplary embodiment. Only G1F was included in determining sequence coverage of Fc/2 subunit.

FIG. 2B shows a comparison of predicted and observed number of fragments of each of low, medium, and high mass sizes for the LC subunit of an antibody using the method of the present invention, according to an exemplary embodiment.

FIG. 2C shows a size distribution of fragments of Fc/2, LC, and Fd subunits of an antibody using settings optimized to produce low, medium, and high mass fragments, according to an exemplary embodiment.

FIG. 3A shows sequence coverage of Fc/2, LC, and Fd subunits of an antibody after combining three independent ETD fragmentations runs for each subunit, according to an exemplary embodiment. FIG. 3A discloses SEQ ID NOS 3-5, respectively, in order of appearance.

FIG. 3B shows sequence coverage of Fc/2, LC, and Fd subunits of an antibody after combining multiple independent ETD fragmentation runs using settings optimized to produce low, medium, and/or high mass fragments, according to an exemplary embodiment. FIG. 3B discloses SEQ ID NOS 3, 3, 3, 4, 4, 4, 5, 5, and 5, respectively, in order of appearance.

FIG. 4 shows an exemplary antibody light and heavy chain sequence of a therapeutic antibody suitable for subjecting to the modeling and assays of the invention. FIG. 4 discloses SEQ ID NOS 1-2, as well as “CPPC” as SEQ ID NO: 6.

### DETAILED DESCRIPTION

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody” refers to a therapeutic immunobinder, e.g., a monoclonal antibody, bi- or multi-specific antibody, that is suitable for introducing into a subject for modulating a disease or disorder, for example, an immune or oncological disorder. The term “antibody” is to be construed broadly as describing monoclonal antibodies, bispecific antibodies, antibody compositions with multi-specificity, as well as antibody fragments or subunits (e.g., Fab, F(ab′)2, scFv, Fv, Fd, Fc/2, and LC), antibody derivatives, fusions, variants, and analogs.

The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the present invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

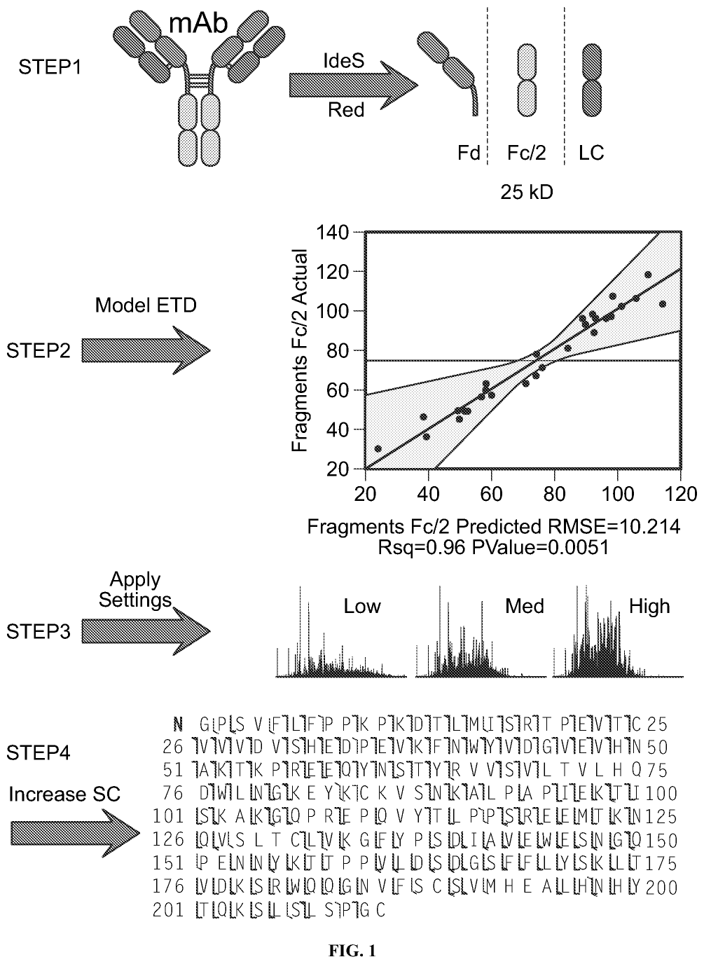

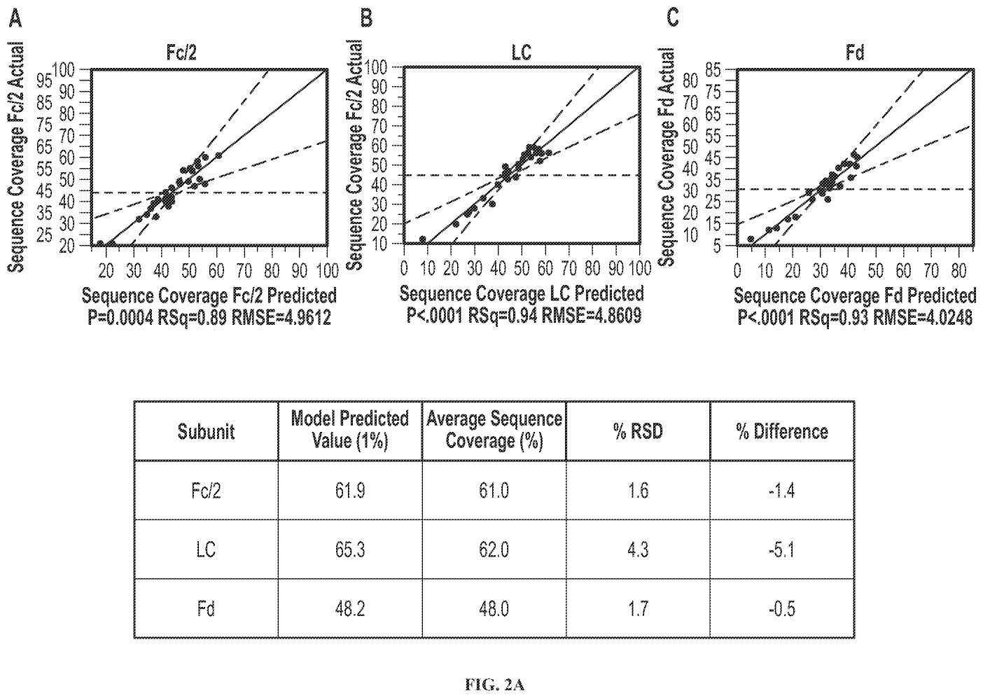

As used herein, the term “Fd” refers to the antibody subunit comprising the heavy chain variable region of an antibody that is approximately 25 kD (see also FIG. 1). As used herein, the term “Fc/2” refers to the antibody subunit comprising the heavy chain constant region of an antibody that is approximately 25 kD (see also FIG. 1). As used herein, the term “LC” refers to the antibody subunit comprising the light chain variable region of an antibody that is approximately 25 kD (see also FIG. 1).

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Muller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

As used herein, the term “NISTmAb” refers to the monoclonal antibody standard “National Institute of Standards & Technology Humanized IgG1κ Monoclonal Antibody standard (NISTmAb)”.

As used herein, the general term “post-translational modifications” or “PTMs” refer to covalent modifications that polypeptides undergo, either during (co-translational modification) or after (post-translational modification) their ribosomal synthesis. PTMs are generally introduced by specific enzymes or enzyme pathways. Many occur at the site of a specific characteristic protein sequence (signature sequence) within the protein backbone. Several hundred PTMs have been recorded, and these modifications invariably influence some aspect of a protein's structure or function (Walsh, G. “Proteins” (2014) second edition, published by Wiley and Sons, Ltd., ISBN: 9780470669853). The various post-translational modifications include, but are not limited to, cleavage, N-terminal extensions, protein degradation, acylation of the N-terminus, biotinylation (acylation of lysine residues with a biotin), amidation of the C-terminal, glycosylation, iodination, covalent attachment of prosthetic groups, acetylation (the addition of an acetyl group, usually at the N-terminus of the protein), alkylation (the addition of an alkyl group (e.g. methyl, ethyl, propyl) usually at lysine or arginine residues), methylation, adenylation, ADP-ribosylation, covalent cross links within, or between, polypeptide chains, sulfonation, prenylation, Vitamin C dependent modifications (proline and lysine hydroxylations and carboxy terminal amidation), Vitamin K dependent modification wherein Vitamin K is a cofactor in the carboxylation of glutamic acid residues resulting in the formation of a γ-carboxyglutamate (a glu residue), glutamylation (covalent linkage of glutamic acid residues), glycylation (covalent linkage glycine residues), glycosylation (addition of a glycosyl group to either asparagine, hydroxylysine, serine, or threonine, resulting in a glycoprotein), isoprenylation (addition of an isoprenoid group such as farnesol and geranylgeraniol), lipoylation (attachment of a lipoate functionality), phosphopantetheinylation (addition of a 4′-phosphopantetheinyl moiety from coenzyme A, as in fatty acid, polyketide, non-ribosomal peptide and leucine biosynthesis), phosphorylation (addition of a phosphate group, usually to serine, tyrosine, threonine or histidine), and sulfation (addition of a sulfate group, usually to a tyrosine residue). The post-translational modifications that change the chemical nature of amino acids include, but are not limited to, citrullination (the conversion of arginine to citrulline by deimination), and deamidation (the conversion of glutamine to glutamic acid or asparagine to aspartic acid). The post-translational modifications that involve structural changes include, but are not limited to, formation of disulfide bridges (covalent linkage of two cysteine amino acids) and proteolytic cleavage (cleavage of a protein at a peptide bond). In an exemplary embodiment, a post-translational modification is cleavage of a lysine at a protein C-terminus. Certain post-translational modifications involve the addition of other proteins or peptides, such as ISGylation (covalent linkage to the ISG15 protein (Interferon-Stimulated Gene)), SUMOylation (covalent linkage to the SUMO protein (Small Ubiquitin-related MOdifier)) and ubiquitination (covalent linkage to the protein ubiquitin). See European Bioinformatics InstituteProtein Information ResourceSlB Swiss Institute of Bioinformatics, European Bioinformatics Institute Drs—Drosomycin precursor—Drosophila melanogaster (Fruit fly)—Drs gene & protein, http://www.uniprot.org/docs/ptmlist for a more detailed controlled vocabulary of PTMs curated by UniProt. In some exemplary embodiments, the method of the present invention may be used to identify, quantify and/or characterize post-translational modifications of a polypeptide, for example an antibody.

As used herein, a “sample” can be obtained from any step of a bioprocess, such as cell culture fluid (CCF), harvested cell culture fluid (HCCF), any step in the downstream processing, drug substance (DS), or a drug product (DP) comprising the final formulated product. In some specific exemplary embodiments, the sample can be selected from any step of the downstream process of clarification, chromatographic production, or filtration.

In some exemplary embodiments, a sample including a protein of interest can be prepared prior to LC-MS analysis. Preparation steps can include denaturation, alkylation, dilution, reduction, and digestion.

As used herein, the term “protein alkylating agent” or “alkylation agent” refers to an agent used for alkylating certain free amino acid residues in a protein. Non-limiting examples of protein alkylating agents are iodoacetamide (IOA/IAA), chloroacetamide (CAA), acrylamide (AA), N-ethylmaleimide (NEM), methyl methanethiosulfonate (MMTS), and 4-vinylpyridine or combinations thereof.

As used herein, “protein denaturing” or “denaturation” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT, or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples of chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion. Digestion of a protein into constituent peptides can produce a “peptide digest” that can further be analyzed using peptide mapping analysis. The term “peptide digest” refers to a peptide mix resultant from exposing a polypeptide, e.g., an antibody, as described herein, when incubated with one or more enzymes (e.g., IdeS) capable of digesting an antibody protein sequence such that polypeptides of appropriate size can be interrogated using the methods of the invention.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

As used herein, the term “protein reducing agent” or “reduction agent” refers to the agent used for reduction of disulfide bridges in a protein. Non-limiting examples of protein reducing agents used to reduce a protein are dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof. A conventional method of protein analysis, reduced peptide mapping, involves protein reduction prior to LC-MS analysis. In contrast, non-reduced peptide mapping omits the sample preparation step of reduction in order to preserve endogenous disulfide bonds. In some exemplary embodiments, a reducing agent is used to separate subunits of an antibody after digestion using IdeS.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed-phase liquid chromatography (RPLC), ion-exchange chromatography, size exclusion chromatography, affinity chromatography, hydrophobic interaction chromatography, hydrophilic interaction chromatography, or mixed-mode chromatography. In some aspects, the sample containing the at least one protein of interest or peptide digest can be subjected to any one of the aforementioned chromatographic methods or a combination thereof. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, UV detection or fluorescence detection.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization (ESI)) or through separate processes. The choice of ion source depends on the application.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS², can be performed by first selecting and isolating a precursor ion (MS¹), and fragmenting it to obtain meaningful information. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers.

Tandem mass spectrometry may produce ion series depending on the fragmentation pattern of the polypeptide analyte, for example, a- and x-ions, b- and y-ions, or c- and z-ions. As used herein, the terms “c-ion” and “z-ion” refer to predominant ions observed when a polypeptide is subjected to the analytical technique ETD.

As used herein, the term “total ion chromatogram” or “total ion current chromatogram” (TIC) refers to a representation of LC-MS data plotting total signal intensity against retention time.

As used herein, the term “top-down” refers to an analytical technique wherein an input sample is a large or intact protein/polypeptide, for example in intact mass analysis. As used herein, the term “bottom-up” refers to an analytical technique wherein an input sample is a protein/polypeptide that has been reduced to small subunits, for example in peptide mapping. As used herein, the term “middle-down” refers to an analytical technique wherein an input sample is a protein/polypeptide that has been reduced to medium sized subunits, for example using digestion of an antibody by IdeS.

As used herein, the term “m/z” or “mass-to-charge ratio” refers to an analytical parameter for characterizing aspects of a polypeptide using, e.g., LC-MS, LC-MS/MS and/or ETD, wherein m stands for mass and z stands for the charge number of ions observed. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein, their abundance, their post-translational modifications or other modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications or sequence variants, or identifying post-translational modifications or sequence variants, or comparability analysis, or combinations thereof.

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary embodiments, the mass analyzer may be a quadrupole mass analyzer, for example a triple quadrupole mass spectrometer. As used herein, the term “RF” refers to the analytical technique for characterizing a polypeptide using radiofrequency (RF) collision quadrupoles.

A mass spectrometer may use one or more of various fragmentation or analysis techniques, including, for example, collision-induced dissociation (CID), electron-transfer dissociation (ETD), electron-transfer/collision-induced dissociation (ETciD), electron-transfer/higher-energy collisional dissociation (EThcD), or ultra-violet photodissociation (UVPD).

In some exemplary embodiments, mass spectrometry analysis may use automatic gain control (AGC). AGC may provide automated regulation to a dynamic ion flux transmitted from the source of the instrument, resulting in a more constant ion population in the mass analyzer to adjust for a broad range of relative abundances in a sample. As used herein, the term “MS² AGC” refers to tandem mass spectrometry analysis with automatic gain control.

In some exemplary embodiments, mass spectrometry can be performed under native conditions. As used herein, the term “native conditions” can include performing mass spectrometry under conditions that preserve non-covalent interactions in an analyte. For a detailed review on native MS, refer to the review: Elisabetta Boeri Erba & Carlo Petosa, The emerging role of native mass spectrometry in characterizing the structure and dynamics of macromolecular complexes, 24 PROTEIN SCIENCE 1176-1192 (2015).

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools.” Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com/proteinpilot), Phenyx (www.phenyx-ms.com), Sorcerer (www.sagenresearch.com), OMS SA (www.pubchem.ncbi.nlm.nih.gov/omssa/), X!Tandem (www.thegpm.org/TANDEM/), Protein Prospector (prospector.ucsfedu/prospector/mshome.htm), Byonic (www.proteinmetrics.com/products/byonic) or Sequest (fields.scripps.edu/sequest).

As used herein, the term “DOE” refers to the design of an experiment that can be facilitated by selected instrument settings, multivariate analysis, and/or computer aided design and software.

As used herein, the term “D-optimal design” refers to a DOE set of parameters suitable for the sequencing of a polypeptide using, for example, ETD and MS. Parameters may include, for example, an m/z isolation window, ETD reaction time, ETD reagent target, and MS² AGC target. When deemed important, parameters affecting ETD MS² spectral quality may be optimized for achieving overall polypeptide fragment/subunit sequence coverage. Parameters may be deemed important based on previous user experience and previous literature. The process includes computer-assisted design, wherein a subset of all relevant combinations is chosen with a goal of maximizing D-efficiency of the design.

As used herein, the term “D-efficiency” refers to computer aided design to decrease workload and provide a meaningful model or D-optimal design of experiment (DOE).

As used herein, the term “THRASH algorithm” refers to analytical software for carrying out aspects of DOE and D-Optimal.

Unless described otherwise, all terms and phrases used herein include the meanings that the terms and phrases have attained in the art, unless the contrary is clearly indicated or clearly apparent from the context in which the term or phrase is used.

This disclosure provides methods for accurately performing high fidelity amino acid residue sequencing of polypeptides, in particular antibody fragments (such as Fd, Fc/2, and LC), at higher rates of precision than have been previously achieved or reported. The assays of the invention are essential quality control tools for evaluating an antibody candidate, for example, in clinical trials or in commercial use.

An exemplary workflow of the method of the invention is illustrated in FIG. 1, where exemplary steps are informed by design of experiment (DOE) input parameters. This DOE informs the application of electron-transfer dissociation (ETD) and mass spectrometry (MS) to give higher sequence coverage (SC) of a given antibody subunit than previously achieved.

The assay of the invention, using a novel set of design of experiment (DOE) parameters, can be calibrated to provide highly accurate measurements. This assay fidelity is key for the manufacture of complex protein molecules, in particular, therapeutic antibodies designed to be introduced into human subjects.

To demonstrate the power of using a DOE approach to antibody sequencing, the method was applied to the National Institute of Standards & Technology Humanized IgG1κ Monoclonal Antibody standard (NISTmAb). By using a DOE approach, MS² parameters were optimized that affect ETD fragmentation and efficiency, and subsequently maximized sequence coverage of NISTmAb monoclonal antibody standard. A combination of three LC-M5² acquisitions using optimal parameters resulted in very high sequence coverages of 71%, 74%, and 58% for Fc/2, LC, and Fd, respectively. It was further demonstrated that the capability of this DOE strategy to model the parameters required to maximize the number of fragments in “low,” “medium,” and “high” m/z ranges resulted in even higher sequence coverages of NISTmAb subunits.

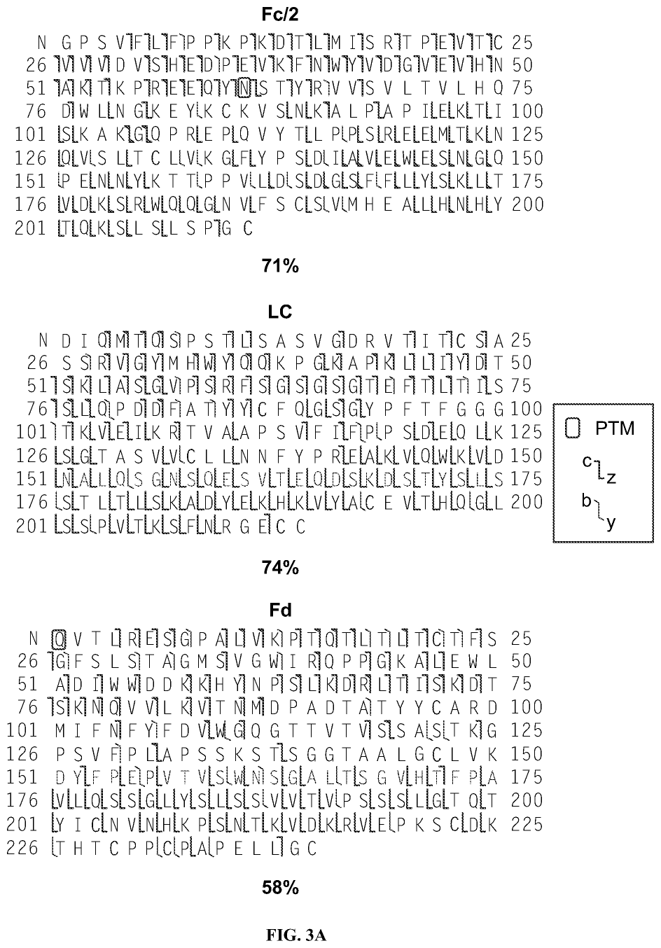

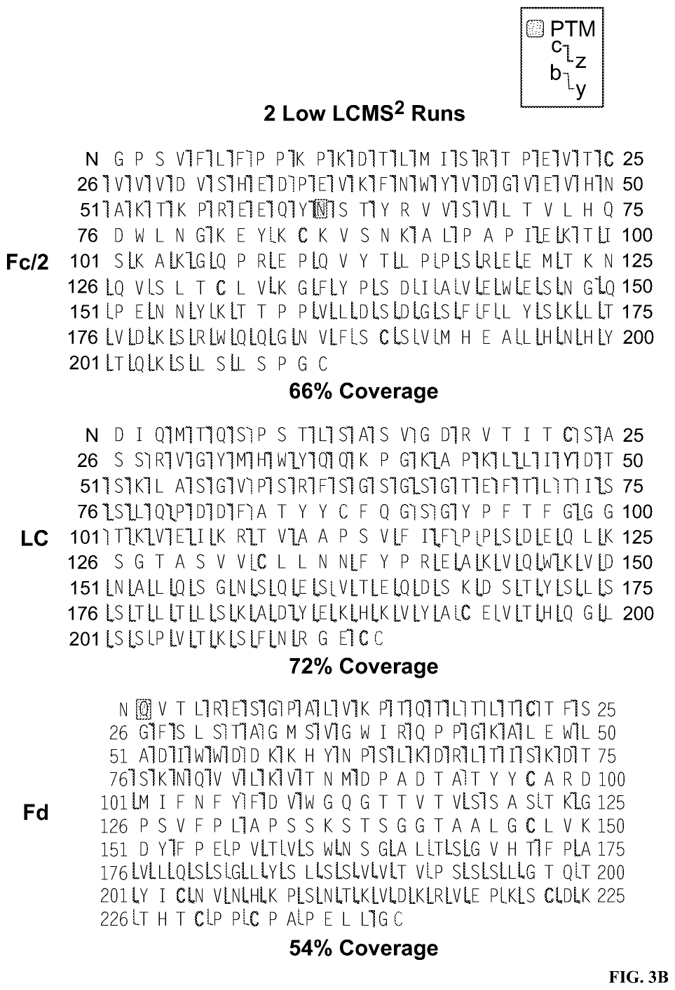

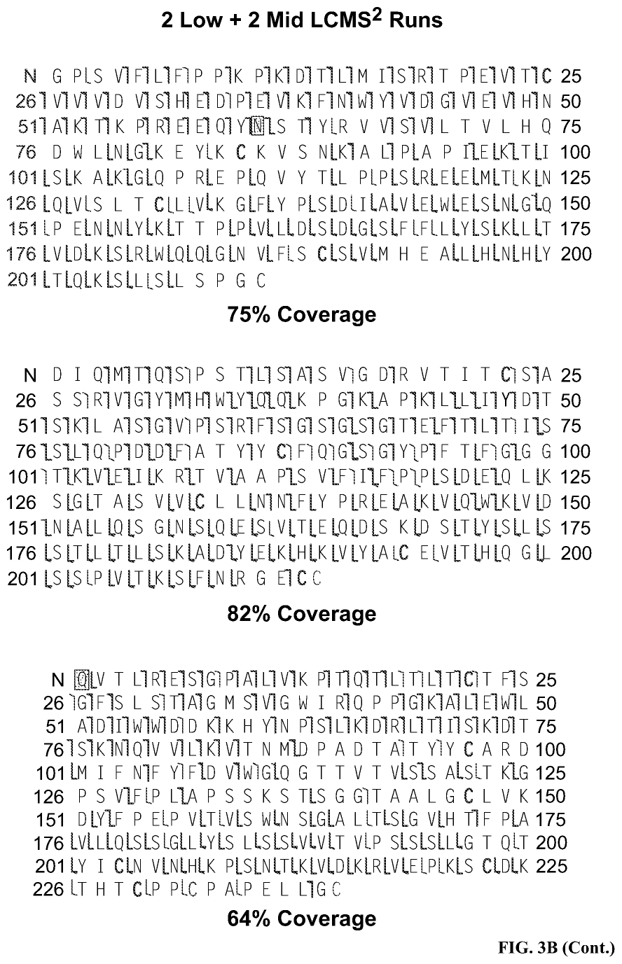

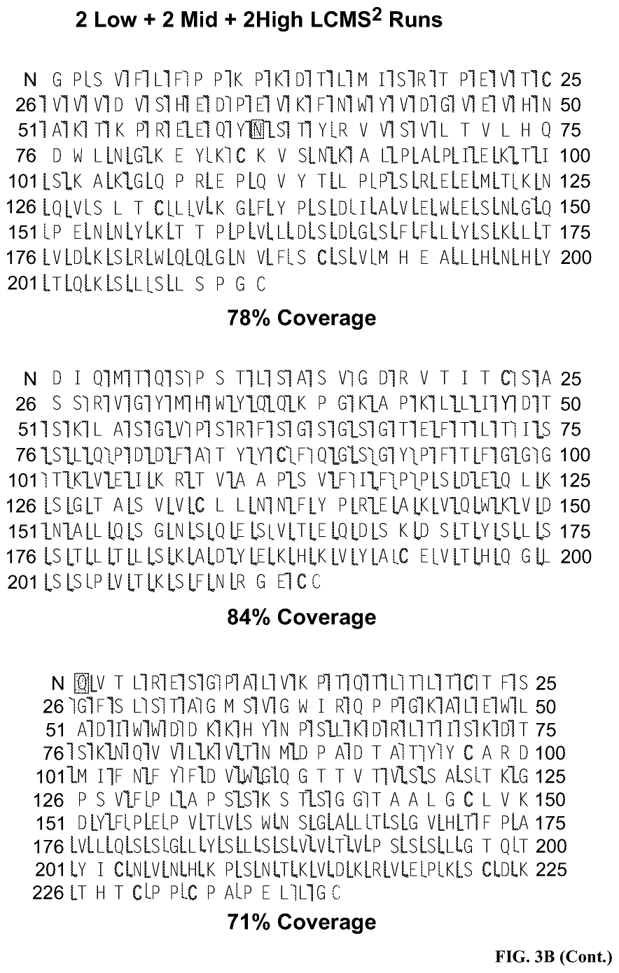

This DOE strategy ultimately resulted in even higher sequence coverages of NISTmAb subunits of 80%, 84%, and 72% for Fc/2, LC, and Fd, respectively, when a total of six runs were combined. These results demonstrate higher ETD sequence coverage of monoclonal antibody subunits when compared to methods previously reported. This approach was also applied to other mAb molecules and similar results were achieved, as shown in FIG. 3A and FIG. 3B.

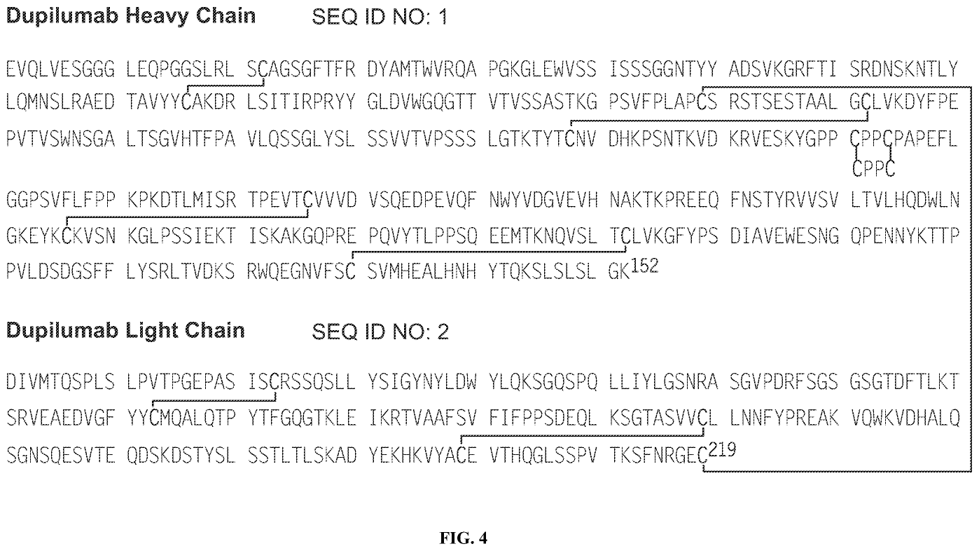

It should be appreciated that the present invention provides for the accurate determination of the fine structure and exact amino acid sequence of a therapeutic antibody while keeping PTMs intact. Accordingly, the invention compliments and improves the CMC (Chemistry, Manufacturing, and Controls) of any commercially produced therapeutic antibody (see, e.g., FIG. 4).

For example, the invention allows for perfecting the manufacture and safeguarding of the homogeneity of a number of antibody therapies. Such antibody therapies include, for example, abciximab, adalimumab, adalimumab-atto, ado-trastuzumab emtansine, alemtuzumab, alirocumab, atezolizumab, avelumab, basiliximab, belimumab, bevacizumab, bezlotoxumab, blinatumomab, brentuximab vedotin, brodalumab, canakinumab, capromab pendetide, certolizumab pegol, cetuximab, daclizumab (Zenapax), daclizumab (Zinbryta), daratumumab, denosumab, dinutuximab, dupilumab, durvalumab, eculizumab, elotuzumab, evolocumab, golimumab, golimumab, ibritumomab tiuxetan, idarucizumab, infliximab, infliximab-abda, infliximab-dyyb, ipilimumab ixekizumab, mepolizumab, natalizumab, necitumumab, nivolumab, obiltoxaximab, obinutuzumab, ocrelizumab, ofatumumab, olaratumab, omalizumab, palivizumab, panitumumab, pembrolizumab, pertuzumab, ramucirumab, ranibizumab, raxibacumab, reslizumab, rituximab, secukinumab, siltuximab, tocilizumab, tocilizumab, trastuzumab, ustekinumab, vedolizumab, sarilumab, rituximab and hyaluronidaseguselkumab, inotuzumab ozogamicin, adalimumab-adbm, gemtuzumab ozogamicin, bevacizumab-awwb, benralizumab, and emicizumab-kxwh. trastuzumab-dkst, infliximab-qbtx, ibalizumab-uiyk, tildrakizumab-asmn, burosumab-twza, and erenumab-aooe.

Other therapeutic antibodies, antibody fragments, antibody fusion proteins, receptors, or receptor proteins of interest for various indications subject to the invention include, for example, aflibercept (e.g., for treating eye disorders); rilonacept (e.g., for treating blindness and metastatic colorectal cancer); alirocumab (e.g., for treating familial hypercholesterolemia or clinical atherosclerotic cardiovascular disease (ASCVD)); dupilumab (e.g., for treating atopic dermatitis); sarilumab (e.g., for treating rheumatoid arthritis and COVID-19); cemiplimab (e.g., for treating PD-1 related disease); and antibodies for treating Ebola.

This disclosure provides a method for sequencing a polypeptide. In some exemplary embodiments, the method comprises exposing the polypeptide to a digest such that a smaller polypeptide sequence is obtained; selecting D-optimal parameters for ETD and MS; subjecting the protein digest to ETD and MS under D-optimal parameters; and determining the amino acid sequence of the polypeptide.

In one aspect, the polypeptide is an antibody, antibody variant, or antibody fusion.

In one aspect, the digest is mediated by the IdeS enzyme. In another aspect, the smaller polypeptide is selected from the group comprising Fd, Fc/2, and LC.

In one aspect, the D-optimal parameters are selected for an analytical chemistry selected from the group comprising ETD, MS, MS¹, MS², MS² AGC, LC-MS, and LC-MS².

In one aspect, the ETD is selected from the group comprising EThcD, ETciD, and UVPD.

In one aspect, the MS is selected from the group comprising MS¹, MS², MS² AGC, LC-MS, and LC-MS².

In one aspect, the determining of the polypeptide amino acid sequence is at least 50%, 60%, 70%, 80%, 90%, and 100%.

This disclosure provides an additional method for sequencing a polypeptide. In some exemplary embodiments, the method comprises exposing the polypeptide to a digest such that a smaller polypeptide sequence is obtained; selecting D-optimal parameters for ETD and LC-MS²; subjecting the protein digest to ETD and LC-MS² under D-optimal parameters; and determining the amino acid sequence of the polypeptide.

In one aspect, the polypeptide is an antibody, antibody variant, or antibody fusion. In a specific aspect, the antibody or antibody fusion is selected from the group comprising aflibercept, rilonacept, alirocumab, dupilumab, sarilumab, cemiplimab, and anti-Ebola antibodies.

This disclosure also provides a polypeptide sequenced according to any of the methods described above.

In one aspect, the polypeptide is selected from the group comprising antibody, antibody variant, and antibody fusion. In another aspect, the polypeptide is selected from the group comprising aflibercept, rilonacept, alirocumab, dupilumab, sarilumab, cemiplimab, and anti-Ebola antibodies.

### EXAMPLES

The examples below are provided for illustrative purposes and should not be construed as limiting the invention which is defined by the appended claims. All references and patents recited within the present application are included herein by reference.

Materials and methods. The present invention, when practiced by the person skilled in the art, may make use of conventional techniques in the field of pharmaceutical chemistry, immunology, molecular biology, cell biology, recombinant DNA technology, and assay techniques, as described in, for example, Sambrook et al. “Molecular Cloning: A Laboratory Manual”, 3rd ed. 2001; Ausubel et al. “Short Protocols in Molecular Biology”, 5th ed. 1995; “Methods in Enzymology”, Academic Press, Inc.; MacPherson, Hames and Taylor (eds.). “PCR 2: A practical approach”, 1995; Harlow and Lane (eds.) “Antibodies, a Laboratory Manual” 1988; Freshney (ed.) “Culture of Animal Cells”, 4th ed. 2000; “Methods in Molecular Biology” vol. 149 (“The ELISA Guidebook” by John Crowther) Humana Press 2001, and later editions of these treatises (e.g., “Molecular Cloning” by Michael Green (4th Ed. 2012) and “Culture of Animal Cells” by Freshney (7th Ed., 2015), as well as current electronic versions.

Methods useful for conducting physical chemistry analysis on peptides and proteins, in particular, antibodies and subunits thereof, are provided within the disclosure as well as described in the following references, and current electronic versions, such as “Introduction to Protein Mass Spectrometry” by Pradip Kumar Ghosh, 2015, “Analytical Characterization of Biotherapeutics” by Jennie R. Lill and Wendy Sandoval, 2017, “Mass Spectrometry of Proteins and Peptides: Methods and Protocols Second Edition (Methods in Molecular Biology)” by Mary S. Lipton and Ljiljana Pas̆a-Tolic, 2008, “Protein Therapeutics, Methods and Principles in Medicinal Chemistry” by Tristan Vaughan, Jane Osbourn, et al., 2017, “Advancements of Mass Spectrometry in Biomedical Research” by Alisa G. Woods and Costel C. Darie, 2019; and “Protein Analysis using Mass Spectrometry: Accelerating Protein Biotherapeutics from Lab to Patient” by Mike S. Lee and Qin C. Ji, 2017.

Reagents. Liquid chromatography-mass spectrometry (LC-MS) grade water, isopropanol (IPA) and acetonitrile (ACN) were purchased from Fisher Chemical (Fair Lawn, N.J.). Formic acid (FA), dithiothreitol (DTT), 8M guanidine-HCl were purchased from Thermo Scientific (Rockford, Ill.). FabRICATOR (IdeS) protease was purchased from Genovis (Cambridge, Mass.).

Sample preparation. NISTmAb, a commercially available humanized IgG1 monoclonal antibody standard, was purchased from MilliporeSigma (St. Louis, Mo.). 100 μg of NISTmAb was digested with 10 units/μL of FabRICATOR protease for 30 minutes at 37° C. After the digestion, 100 μL of guanidine-HCl and 25 μL of 1.0 M DTT was added, and the sample was incubated at 37 ° C. for 45 minutes to reduce the antibody to Fd, Fc/2, and LC subunits.

Liquid chromatography. Reversed-phase liquid chromatography (RPLC) was performed on an I-class UPLC® instrument equipped with a binary solvent manager (BSM) from Waters Corp. (Milford, Mass.). Prior to MS analysis, all samples were desalted using a Protein BEH C4 VanGuard™ column from Waters Corp. (Milford, Mass.). Following the desalting step, the fragments were separated using a 1 mm×150 mm Protein BEH C4 (300 Å, 1.7 μ.m) column from Waters Corp. Mobile phase A (MPA) was 0.1% FA in water and mobile phase B (MPB) was 60% ACN, 39.9% IPA and 0.1% FA.

For each experiment, 1 μg of digested and reduced antibody was loaded onto a column. Following sample loading, the samples were desalted at 5% MPB for five minutes. The gradient was then increased to 20% MPB over two minutes, followed by an increase to 60% MPB over 28 minutes. Flow rate was 100 μL/min. Following protein elution, the column was restored to 5% MPB prior to next sample injection.

Mass spectrometry. An I-class instrument was coupled to an ESI source of a Thermo Scientific Fusion Lumos Tribrid mass spectrometer equipped with an ETD reagent source located in the front-end of the instrument. Spray voltage was set to 3700 V and ion transfer tube temperature was set to 350° C. Prior to design of experiments (DOE) runs, three MS¹ experiments were performed to determine the elution window and charge distribution for each subunit, and to ensure that there is minimal retention time shift between the runs.

Information from the three MS' experiments was used to create different targeted MS² based on the DOE. For the MS² experiments, Orbitrap resolution was set to 120,000, the mass range was set to normal (350-2000 m/z) and the RF lens percentage was set to 30. Each MS² scan was a composite of 10 microscans. Quadrupole was used for precursor isolation and the isolation window was centered at the most intense charge state based on the initial MS¹ experiments.

Data analysis. EVP statistical software was used to generate different DOE runs based on the chosen factors. For each run, total ion chromatograms (TICs) were initially evaluated in Xcalibur to determine scan ranges for each subunit. THRASH algorithm in ProSight PC 4.0 was then used to deconvolute isotopic clusters in the MS² spectra for each scan range determined from Xcalibur. THRASH parameters in ProSight PC were set as following: minimum signal-to-noise ratio was set to 8 and minimum RL (reliability) value was set 0.95. MS² fragment mass tolerance was set to 20 ppm.

Following deconvolution, ProSight results were exported to ProSight Lite where appropriate post-translational modifications (PTMs) were added to the subunit sequence and total subunit coverage determined based on the MS² fragments. The sequence coverage results were then exported to JMP statistical software where modeling was performed for each subunit to determine the relationship between the chosen factors and measured responses.

The following working examples demonstrate exemplary methods for improved amino acid sequencing of polypeptides, in particular, antibody subunits.

### Example 1. Design of Experiment (DOE) Strategy

This example describes the design of experiment (DOE) considerations for optimizing electron transfer dissociation (ETD) parameters for the accurate sequencing of antibody subunits.

First, previous knowledge and experience, and information from the published literature, was used to determine the ETD MS² factors to evaluate and to set the bounds of the design space. Setting bounds that are outside the design space were excluded as not providing meaningful relationships between the factors and responses. It should be understood that parameters that affect the pertinent outcome, for example sequence coverage or the number of identified fragments of a given size, may be selected by a person of skill in the art.

The chosen factors deemed relevant for ETD MS² sequence coverage or the number of identified fragments included the isolation window, ETD reaction time, ETD reagent target, and the MS² AGC target. To determine whether there is a curvilinear relationship between the factors, each factor was evaluated at three different levels. The isolation window was evaluated at 100, 400 and 700 m/z. The ETD reaction time was evaluated at 5, 10 and 15 ms. The ETD reagent target was evaluated at 1.0E5, 5.5E5 and 1.0E6. The MS² AGC target was evaluated at 5.0E4, 5.3E5 and 1.0E6.

To evaluate relationships between the factors impacting the ETD MS² spectra, one possible approach is to proceed with a full factorial DOE. However, with the number of factors and levels chosen, a full factorial DOE would require 81 runs, taking up significant resources in terms of run time and data analysis. Instead, a more efficient approach was chosen using a D-optimal DOE, which only required 28 UPLC-MS runs to maximize the D-efficiency parameter of the DOE.

Accordingly, this example represents aspects of the invention that illustrate the value of optimizing DOE parameters for ease of use in sequencing a polypeptide while maintaining accuracy and sequence coverage.

### Example 2. Optimal Conditions from Predictive Models to Generate Maximum Sequence Coverage

This example describes exemplary optimal assay conditions for generating maximum amino acid residue coverage of an antibody subunit.

A first goal was to find an optimal set of conditions using DOE that would maximize ETD middle-down sequence coverage in a single run. For the ETD optimization, an evaluate isolation window, ETD reaction time, ETD reagent target, and MS² AGC target were selected.

Isolation window, which controls the m/z range of ions sent to the linear ion trap for MS² ETD, was centered at the most intense charge state observed in the MS¹ spectra for each subunit.

Electron transfer dissociation reaction time refers to the amount of time that fluoranthene anions react with precursor ions, and ETD reagent target refers to the amount of fluoranthene anions that are injected into the linear ion trap and allowed to react with the precursor ions.

Observations showed that the parameters mentioned are directly responsible for the quality of MS² ETD spectra and would therefore have an impact on the MS² sequence coverage. Initially, a JMP statistical software was used to determine a subset from all possible combinations of parameters chosen.

To maximize the D-efficiency of these models, a total of 28 runs were collected for each DOE performed, and parameters impacting ETD MS² spectra of each subunit were modeled separately to determine the optimal parameter settings that would provide maximum sequence coverage.

After processing the data using the THRASH algorithm in ProSight PC, the results were exported to ProSight Lite where post-translational modifications, such as N-linked glycosylation of the Fc/2 subunit and conversion of N-terminal glutamine to pyroglutamic acid of the Fd subunit, were recorded. For Fc/2, because G1F is the most abundant glycoform of NISTmAb, only G1F species were included in calculating sequence coverage. For Fd, the sequence with pyroglutamic acid was considered because most glutamine is rapidly converted to pyroglutamic acid and would therefore be considered a major species.

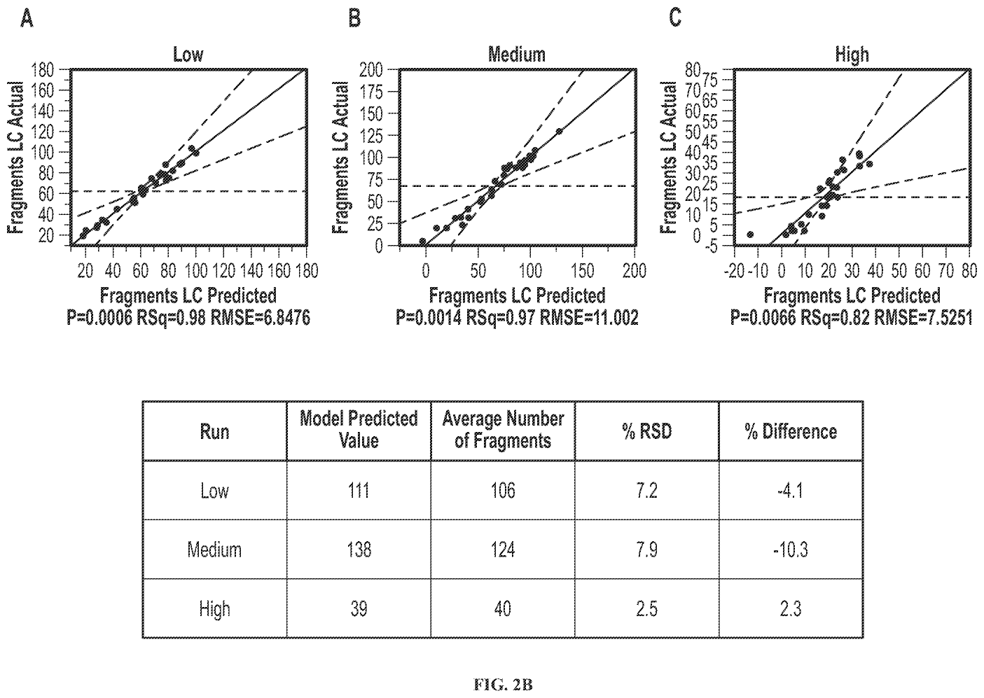

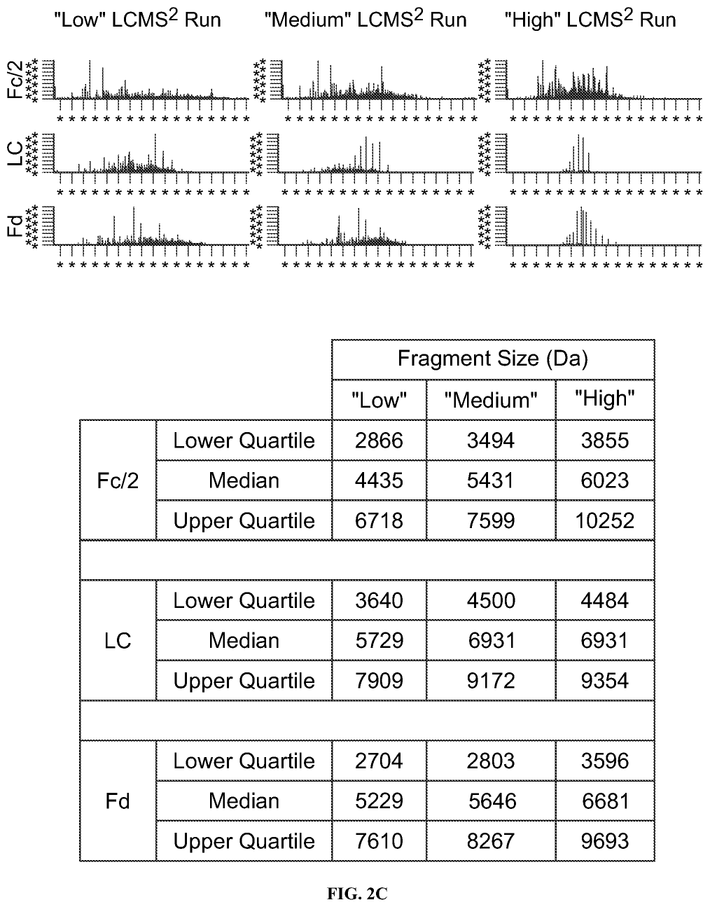

Based on the results of the DOE, optimal Fc/2 parameters were found to be 539.8 m/z, 15 ms, 9.2E5 and 9.5E5 for isolation width, ETD reaction time, ETD reagent target and MS² AGC target, respectively. The model for Fc/2 shows strong correlation between model-predicted sequence coverage and actual sequence coverage observed, as shown in FIG. 2A, Panel A. The average middle-down sequence coverage of three runs using optimal ETD parameters was 61.0% for the Fc/2 subunit.

DOE models showed that 397.1 m/z, 15 ms, 9.3E5 and 1E6 for isolation width, ETD reaction time, ETD reagent target and MS² AGC target, respectively, were the optimal operating parameters to achieve maximum sequence coverage of NISTmAb light chain (LC), as shown in FIG. 2A, Panel B. When three runs using parameters for optimal ETD fragmentation were averaged, it was observed that 62.0% sequence coverage of the light chain was achieved.

Lastly, for the Fd subunit, isolation width of 503.6 m/z, ETD reaction time of 15 ms, ETD reagent target 7.9E5 and MS² AGC target of 8.0E5 were determined to be the optimal operating parameters for maximum sequence coverage using ETD, as shown in FIG. 2A, Panel C.

Upon determining the optimal parameters for maximum sequence coverage from the DOE models, an experiment in triplicate was performed using the parameters to determine how the model-predicted values compared to the actual values. Actual experiment values show strong correlation with the model predicted values, with minimal percent error observed, as shown in FIG. 2A.

Accordingly, this example demonstrates that the DOE strategy of the present invention resulted in the derivation of optimal parameter settings for maximizing amino acid sequence coverage of a polypeptide, for example an antibody subunit, that corresponded well with observed sequence coverage.

### Example 3. Combined Sequence Coverage from Multiple LC-MS Runs

This example describes exemplary assay conditions for generating maximum amino acids residue sequence coverage of a polypeptide, for example, an antibody fragment.

It has been shown that combining ETD fragmentation runs from multiple MS² runs can further increase sequence coverage of monoclonal antibody subunits (Fornelli et al. 2018; Fornelli et al. 2012). After determining the optimal ETD parameters for maximum sequence coverage using the DOE strategy of the present invention, identified fragments from three independent ETD RPLC-MS² runs were combined to determine the combined sequence coverage of each subunit. This approach resulted in a 10-12% increase of sequence coverage for Fc/2, LC, and Fd subunits, as shown in FIG. 3A.

Accordingly, this example demonstrates that the method of the present invention can be used to further improve antibody sequence coverage by combining identified fragments from independent ETD LC-MS experiments.

### Example 4. Obtaining Maximum Numbers of Fragments of Low, Medium and High Mass for Improved Antibody Subunit Sequence Coverage

This example demonstrates a novel method for designing assay conditions for generating maximum amino acid residue sequence coverage of a polypeptide, for example an antibody subunit.

Initial DOE findings revealed that the majority of c- and z-ions produced by the optimized ETD parameters tended to concentrate closer to the N- and C-termini of each mAb subunit. Based on these findings, a new approach was designed to maximize sequence coverage of each subunit by creating DOE models that determine ETD MS² parameters capable of generating different size fragments, for example low-, medium- and high-mass size fragments. For these experiments, low mass was defined as fragments below 5,000 Da, medium mass was defined as fragments between 5,000 Da and 10,000 Da, and high mass was defined as fragments larger than 10,000 Da. Combining the results from RPLC-MS² runs obtained using the low-, medium-, and high-mass conditions could potentially increase the sequence coverage as well as the fragment size diversity. This approach generated predictive DOE models measuring the number of ETD MS² fragments generated for each mass region. On the bases of the DOE results, the ETD parameters that generated the greatest number of low-, medium-, and high-mass fragments for the three subunits were determined. FIG. 2B shows the DOE models for low-, medium-, and high-mass conditions, demonstrating strong correlations between the actual and model-predicted number of fragments as well as a high model significance. In addition, the mean number of fragments observed experimentally when using the optimal ETD MS² parameters for each fragment size category was comparable to those predicted by each model.

The examination of raw MS² spectra produced using the DOE models revealed dramatic differences among the spectral profiles. For Fc/2, LC, and Fd, the mass distribution of fragments shifted depending on the ETD MS² parameters used, as shown in FIG. 2C. In the case of Fc/2, an increase in the lower and upper quartiles as well as median fragment mass was observed for the low-, medium-, and high-mass conditions. A similar but less pronounced trend was observed for Fd even with the previously documented difficulties in achieving high Fd sequence coverage using ETD. For the LC subunit, a substantial difference was observed in lower and upper quartiles as well as median fragment mass between low- and medium-mass conditions, but only a small difference was observed between medium- and high-mass conditions. Regardless, the high-mass ETD MS² parameters provided additional high-mass fragments and further increased sequence coverage information on the LC subunit.

This example demonstrates that improved sequence coverage can be obtained using the novel method of DOE optimization for obtaining different sized fragments of a polypeptide using ETD M5², for example, low-, medium-, and high-mass fragments.

### Example 5. Combined Sequence Coverage from Low-, Medium-, and High-Mass Conditions

The shifting median fragment mass observed for Fc/2, LC, and Fd subunits using low-, medium-, and high-mass conditions as shown in FIG. 2C indicates that these different conditions generated significantly different ion populations, which when combined may provide increased sequence coverage information on the mAb subunits. Therefore, the method of the present invention was further optimized by combining the fragmentation results from low-, medium-, and high-mass ETD RPLC-MS² runs to gauge the overall increase in sequence coverage for each subunit.

Two low-mass, two medium-mass, and two high-mass RPLC-MS² runs were combined for a total of six independent runs using ETD parameters determined from the fragment mass DOE as previously described. Each subsequent addition of fragmentation data from two RPLC-MS² runs further increased the sequence coverage of each subunit. This approach resulted in improved sequence coverages for Fc/2, LC, and Fd, as shown in FIG. 3B. The addition of fragments from nonglycosylated Fc/2 as well as all abundant glycoforms further increased the sequence coverage for Fc/2. These sequence coverage results obtained with ETD alone compare favorably to a previous report using multiple RPLC-MS² runs and six different ETD reaction times. Combining fragmentation data from more than six RPLC-MS² runs resulted in slight or no sequence coverage gain at the expense of instrument and data analysis time.

A comprehensive workflow for optimization of ETD MS² instrument operating parameters to maximize sequence coverage of monoclonal antibody subunits was developed. Two distinct approaches were employed for maximizing sequence coverage. The first method determined optimal ETD MS² parameters through DOE models to maximize sequence coverage. The second method improved sequence coverage by determining ETD conditions that produce fragments in low-, medium-, and high-mass ranges. Using this method and combining sequence coverages of Fc/2, LC, and Fd subunits yielded an improved sequence coverage for the entire mAb.

The DOE approaches described here can be used to generate predictive models that increase middle-down sequence coverage for mAbs across different mass spectrometer platforms without requiring additional fragmentation techniques. This approach could be extended to other fragmentation methods including EThcD, ETciD, and UVPD to further improve amino acid sequence coverage and achieve even closer to complete sequence coverage of a monoclonal antibody or other polypeptide.

While in the foregoing specification this invention has been described in relation to certain embodiments thereof, and many details have been put forth for the purpose of illustration, it will be apparent to those skilled in the art that the invention is susceptible to additional embodiments and that certain details described herein can be varied without departing from the basic principles of the invention.

What is claimed is:

1. A method for improving sequence coverage of a polypeptide, comprising:
(a) selecting at least two parameters for tandem mass spectrometry that affect sequence coverage; and
(b) using D-optimal design of experiments to determine a value of each of said at least two parameters, wherein said value is selected based on maximizing sequence coverage.

2. The method of claim 1, wherein said polypeptide is an antibody, a bispecific antibody, a monoclonal antibody, a fusion protein, an antibody-drug conjugate, an antibody fragment, an antibody subunit, a host cell protein, a protein pharmaceutical product, or a digested fragment thereof.

3. The method of claim 1, further comprising carrying out the method in sequence or in parallel for two or more subunits of a polypeptide.

4. The method of claim 3, wherein said subunits are selected from a group including an Fc/2, Fd, or LC subunit of an antibody.

5. The method of claim 1, wherein said tandem mass spectrometry is middle-down mass spectrometry.

6. The method of claim 1, wherein said mass spectrometer is an electrospray ionization mass spectrometer, nano-electrospray ionization mass spectrometer, or an Orbitrap-based mass spectrometer.

7. The method of claim 1, wherein said tandem mass spectrometry includes electron-transfer dissociation, collision-induced dissociation, electron-transfer/collision-induced dissociation, electron-transfer/higher-energy collisional dissociation, ultra-violet photodissociation, or a combination thereof.

8. The method of claim 1, wherein said tandem mass spectrometry includes automatic gain control.

9. The method of claim 1, wherein said mass spectrometer is coupled to a liquid chromatography system.

10. The method of claim 9, wherein said liquid chromatography system comprises reversed-phase liquid chromatography, ion exchange chromatography, size exclusion chromatography, affinity chromatography, hydrophobic interaction chromatography, hydrophilic interaction chromatography, mixed-mode chromatography, or a combination thereof.

11. The method of claim 1, wherein said at least two parameters are selected from a group including m/z isolation window, ETD reaction time, ETD reagent target, MS² AGC target, and any combination thereof.

12. A method for determining an amino acid sequence of a polypeptide, comprising:
(a) determining a value of at least two parameters for tandem mass spectrometry for a polypeptide using D-optimal design of experiments, wherein said value is selected based on maximizing sequence coverage; and
(b) subjecting said polypeptide to tandem mass spectrometry analysis using said values of said at least two parameters to determine an amino acid sequence of said polypeptide.

13. The method of claim 12, wherein said polypeptide is an antibody, a bispecific antibody, a monoclonal antibody, a fusion protein, an antibody-drug conjugate, an antibody fragment, an antibody subunit, a host cell protein, a protein pharmaceutical product, or a digested fragment thereof.

14. The method of claim 12, further comprising carrying out the method in sequence or in parallel for two or more subunits of a polypeptide.

15. The method of claim 14, wherein said subunits are selected from a group including an Fc/2, Fd, or LC subunit of an antibody.

16. The method of claim 12, wherein said tandem mass spectrometry is middle-down mass spectrometry.

17. The method of claim 12, wherein said mass spectrometer is an electrospray ionization mass spectrometer, nano-electrospray ionization mass spectrometer, or an Orbitrap-based mass spectrometer.

18. The method of claim 12, wherein said tandem mass spectrometry includes electron-transfer dissociation, collision-induced dissociation, electron-transfer/collision-induced dissociation, electron-transfer/higher-energy collisional dissociation, ultra-violet photodissociation, or a combination thereof.

19. The method of claim 12, wherein said tandem mass spectrometry includes automatic gain control.

20. The method of claim 12, wherein said mass spectrometer is coupled to a liquid chromatography system.

21. The method of claim 20, wherein said liquid chromatography system comprises reversed-phase liquid chromatography, ion exchange chromatography, size exclusion chromatography, affinity chromatography, hydrophobic interaction chromatography, hydrophilic interaction chromatography, mixed-mode chromatography, or a combination thereof.

22. The method of claim 12, wherein said parameters are selected from a group including m/z isolation window, ETD reaction time, ETD reagent target, MS² AGC target, and any combination thereof.

23. The method of claim 12, further comprising subjecting said polypeptide to enzymatic digestion prior to tandem mass spectrometry analysis.

24. The method of claim 23, wherein said enzymatic digestion comprises contacting said polypeptide to IdeS.

25. The method of claim 12, further comprising subjecting said polypeptide to reduction prior to tandem mass spectrometry analysis.

26. The method of claim 12, further comprising carrying out the method independently two or more times and combining identified fragments from each tandem mass spectrometry analysis to determine an amino acid sequence of said polypeptide.

27. A method for improving sequence coverage of a polypeptide, comprising:
(a) selecting at least two parameters for tandem mass spectrometry that affect the number of fragments of each of a selection of different fragment sizes; and
(b) using D-optimal design of experiments to determine a value of each of said at least two parameters, wherein said value is selected based on producing the greatest number of fragments for each of said selected fragment sizes.

28. The method of claim 27, wherein said polypeptide is an antibody, a bispecific antibody, a monoclonal antibody, a fusion protein, an antibody-drug conjugate, an antibody fragment, an antibody subunit, a host cell protein, a protein pharmaceutical product, or a digested fragment thereof.

29. The method of claim 27, further comprising carrying out the method in sequence or in parallel for two or more subunits of a polypeptide.

30. The method of claim 29, wherein said subunits are selected from a group including an Fc/2, Fd, or LC subunit of an antibody.

31. The method of claim 27, wherein said tandem mass spectrometry is middle-down mass spectrometry.

32. The method of claim 27, wherein said mass spectrometer is an electrospray ionization mass spectrometer, nano-electrospray ionization mass spectrometer, or an Orbitrap-based mass spectrometer.

33. The method of claim 27, wherein said tandem mass spectrometry includes electron-transfer dissociation, collision-induced dissociation, electron-transfer/collision-induced dissociation, electron-transfer/higher-energy collisional dissociation, ultra-violet photodissociation, or a combination thereof.

34. The method of claim 27, wherein said tandem mass spectrometry includes automatic gain control.

35. The method of claim 27, wherein said mass spectrometer is coupled to a liquid chromatography system.

36. The method of claim 35, wherein said liquid chromatography system comprises reversed-phase liquid chromatography, ion exchange chromatography, size exclusion chromatography, affinity chromatography, hydrophobic interaction chromatography, hydrophilic interaction chromatography, mixed-mode chromatography, or a combination thereof.

37. The method of claim 27, wherein said at least two parameters are selected from a group including m/z isolation window, ETD reaction time, ETD reagent target, MS² AGC target, and any combination thereof.

38. The method of claim 27, wherein said fragment sizes are selected from a group including fragments below about 5,000 Da, fragments between about 5,000 Da and about 10,000 Da, and fragments larger than about 10,000 Da.

39. A method for determining an amino acid sequence of a polypeptide, comprising:
(a) determining a value of at least two parameters for tandem mass spectrometry for a polypeptide using D-optimal design of experiments, wherein said value is selected based on producing the greatest number of fragments for each of a selection of different fragment sizes;
(b) subjecting said polypeptide to tandem mass spectrometry analysis using said values of said at least two parameters for each selected fragment size; and
(c) combining identified fragments from said tandem mass spectrometry analysis using said values of said at least two parameters for each selected fragment size to determine an amino acid sequence of said polypeptide.

40. The method of claim 39, wherein said polypeptide is an antibody, a bispecific antibody, a monoclonal antibody, a fusion protein, an antibody-drug conjugate, an antibody fragment, an antibody subunit, a host cell protein, a protein pharmaceutical product, or a digested fragment thereof.

41. The method of claim 39, further comprising carrying out the method in sequence or in parallel for two or more subunits of a polypeptide.

42. The method of claim 41, wherein said subunits are selected from a group including an Fc/2, Fd, or LC subunit of an antibody.

43. The method of claim 39, wherein said tandem mass spectrometry is middle-down mass spectrometry.

44. The method of claim 39, wherein said mass spectrometer is an electrospray ionization mass spectrometer, nano-electrospray ionization mass spectrometer, or an Orbitrap-based mass spectrometer.

45. The method of claim 39, wherein said tandem mass spectrometry includes electron-transfer dissociation, collision-induced dissociation, electron-transfer/collision-induced dissociation, electron-transfer/higher-energy collisional dissociation, ultra-violet photodissociation, or a combination thereof.

46. The method of claim 39, wherein said tandem mass spectrometry includes automatic gain control.

47. The method of claim 39, wherein said mass spectrometer is coupled to a liquid chromatography system.

48. The method of claim 47, wherein said liquid chromatography system comprises reversed-phase liquid chromatography, ion exchange chromatography, size exclusion chromatography, affinity chromatography, hydrophobic interaction chromatography, hydrophilic interaction chromatography, mixed-mode chromatography, or a combination thereof.

49. The method of claim 39, wherein said at least two parameters are selected from a group including m/z isolation window, ETD reaction time, ETD reagent target, MS² AGC target, and any combination thereof.

50. The method of claim 39, further comprising subjecting said polypeptide to enzymatic digestion prior to tandem mass spectrometry analysis.

51. The method of claim 50, wherein said enzymatic digestion comprises contacting said polypeptide to IdeS.

52. The method of claim 39, further comprising subjecting said polypeptide to reduction prior to tandem mass spectrometry analysis.

53. The method of claim 39, further comprising carrying out the method independently two or more times and combining identified fragments from each tandem mass spectrometry analysis to determine an amino acid sequence of said polypeptide.

54. A method for determining an amino acid sequence of an antibody, comprising:
(a) selecting at least two parameters for ETD-MS² that affect sequence coverage for each subunit of an antibody, wherein said subunits include Fd, Fc/2 and LC;
(b) using D-optimal design of experiments to determine a value of each of said at least two parameters for each of said subunits, wherein said value is selected based on maximizing sequence coverage of said subunit;
(c) contacting said antibody to IdeS and a reducing agent to produce said subunits;
(d) subjecting each of said subunits to ETD-MS² analysis using said values of said at least two parameters to identify amino acid sequences of fragments of each of said subunits;
(e) independently repeating step (d) at least one more time to identify amino acid sequences of additional fragments of each of said subunits; and
(f) combining said amino acid sequences of said fragments of (d) and (e) to determine an amino acid sequence of said antibody.

55. A method for determining an amino acid sequence of an antibody, comprising:
(a) selecting at least two parameters for ETD-MS² that affect the number of small, medium, and large fragments of each subunit of an antibody, wherein said subunits include Fd, Fc/2 and LC, said small fragments consist of fragments smaller than about 5,000 Da, said medium fragments consist of fragments between about 5,000 Da and about 10,000 Da, and said large fragments consist of fragments greater than 10,000 Da;
(b) using D-optimal design of experiments to determine a value of each of said at least two parameters for each of said fragment sizes for each of said subunits, wherein said value is selected based on producing the greatest number of fragments of said size for said subunit;
(c) contacting said antibody to IdeS and a reducing agent to produce said subunits;
(d) subjecting each of said subunits to ETD-MS² analysis using said values of said at least two parameters for each of said fragment sizes to identify amino acid sequences of fragments of each of said subunits;
(e) independently repeating step (d) at least one more time to identify amino acid sequences of additional fragments of each of said subunits; and
(f) combining said amino acid sequences of said fragments of (d) and (e) to determine an amino acid sequence of said antibody.


SEQUENCE LISTING (6 sequences)

SEQ ID NO: 1 - protein, 453 aa, Artificial Sequence
  Description of Artificial Sequence Synthetic polypeptide
EVQLVESGGGLEQPGGSLRLSCAGSGFTFRDYAMTWVRQAPGKGLEWVSSISSSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKITTPPVLDSDGSFFLYSRLTVDKSRWQEGNVFSCSVMHEALHNHYTQKSLSLSLGK

SEQ ID NO: 2 - protein, 219 aa, Artificial Sequence
  Description of Artificial Sequence Synthetic polypeptide
DIVMTQSPLSLPVTPGEPASISCRSSQSLLYSIGYNYLDWYLQKSGQSPQLLIYLGSNRASGVPDRFSGSGSGTDFTLKTSRVEAEDVGFYYCMQALQTPYTFGQGTKLEIKRTVAAFSVFIFPPSDEQLKSGTASVVCLLNNFYPREAKVQWKVDHALQSGNSQESVTEQDSKDSTYSLSSTLTLSKADYEKHKVYACEVTHQGLSSPVTKSFNRGEC

SEQ ID NO: 3 - protein, 210 aa, Artificial Sequence
  Description of Artificial Sequence Synthetic polypeptide
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 213 aa, Artificial Sequence
  Description of Artificial Sequence Synthetic polypeptide
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 239 aa, Artificial Sequence
  Description of Artificial Sequence Synthetic polypeptide
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLG

SEQ ID NO: 6 - protein, 4 aa, Artificial Sequence
  Description of Artificial Sequence Synthetic peptide
CPPC